(19) United States
(12) Patent Application Publication
(10) Pub. No.: US 2024/0180903 A1
(43) Pub. Date: Jun. 6, 2024

(54) INJECTABLE PHARMACEUTICAL COMPOSITION, PREPARATION METHOD THEREFOR AND USE THEREOF

(71) Applicant: ZHEJIANG CUIZE PHARMACEUTICAL TECHNOLOGY CO., LTD., Hangzhou (CN)

(72) Inventors: Yi SHEN, Hangzhou (CN); Qiang GE, Hangzhou (CN); Siping SUN, Hangzhou (CN); Dingqiang JIANG, Hangzhou (CN); Cong YE, Hangzhou (CN)

(21) Appl. No.: 18/186,250

(22) Filed: Mar. 20, 2023

(30) Foreign Application Priority Data
Dec. 1, 2022 (CN) 202211538523.2

(51) Int. Cl. A61K 31/4985; A61K 9/00; A61K 9/10; A61K 47/10; A61K 47/14; A61K 47/26; A61K 47/38

(52) U.S. Cl. CPC A61K 31/4985; A61K 9/0019; A61K 9/10; A61K 47/10; A61K 47/14; A61K 47/26; A61K 47/38

(57) ABSTRACT

An injectable pharmaceutical composition, a preparation method therefor and use thereof are provided. The pharmaceutical composition contains an active ingredient and a pharmaceutically acceptable auxiliary material. The active ingredient comprises cabotegravir or a pharmaceutically acceptable salt thereof, and the auxiliary material comprises a surfactant and a suspending agent. The pharmaceutical composition and a formulation thereof are used for treating and preventing HIV infection, have good stability, and are suitable for industrial production.

### TECHNICAL FIELD

The present disclosure relates to the field of pharmaceutical formulations, and particularly relates to an injectable pharmaceutical composition comprising cabotegravir, a preparation method therefor and use thereof.

### BACKGROUND

Acquired immunodeficiency syndrome (AIDS) is caused by human immunodeficiency virus (HIV). HIV is feared in that it treats the most important CD4 T lymphocytes in the human immune system as the main attack target, destroying these cells in large quantities, and causing the loss of immune function in the human body. Treatment compliance and stigma remain major obstacles to combating the HIV-1 epidemic. In China, AIDS has entered a rapid growth period, and the epidemic development trend is sharp. Therefore, the search for an effective method for treating AIDS has become a top priority and a hot spot in the world.

Cabotegravir (formula I) is the 2nd generation integrase strand transfer inhibitor (INSTI) produced by ViiV Healthcare, the special pharmaceutical properties of which make it suitable for formulating into a long-acting sustained-release formulation.

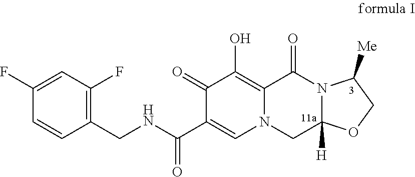 [SMILES: C[C@H]1CO[C@@H]2Cn3cc(C(=O)NCc4ccc(F)cc4F)c(=O)c(O)c3C(=O)N12]

Cabotegravir has a strong efficacy with IC₅₀ against HIV of 0.22 nmol/L. Cabotegravir can be orally administered at a dose of 5 mg/d or 30 mg/d, and HIV RNA in plasma is reduced to 2.2-2.3 copies/mL 10 d after a single dose. Cabotegravir can also be administered by injection with an increased half-life and longer dosing intervals, thereby avoiding the trouble of daily oral administration. The metabolites of cabotegravir are mainly excreted out of the body through bile and urine, so its processes in vivo will not be affected by other drugs. Cabenuva, developed by ViiV Healthcare, is a two-vial formulation containing a vial of cabotegravir suspension and a vial of rilpivirine suspension (i.e., a controlled-release long-acting depot administration system made of nanosuspensions), which is administered by intramuscular injection. The plasma concentration of the drug can be maintained at more than 4 times the 90% inhibitory concentration (IC₉₀) by intramuscular injection once every month or once every two months.

According to the production process described in WO2012/037320 published by ViiV Healthcare, in order to prepare a nanoparticle composition by co-grinding the pharmaceutical composition containing cabotegravir, the production process is relatively complicated, the requirements for production equipment are relatively high, and the expansion of the yield is also limited. In addition, in order to ensure the stability of the nanosuspension system, the storage conditions of the product should be controlled at 2-8° C.

Therefore, there is an urgent need to develop a novel formulation of cabotegravir that is stable, easy to store, and/or simple to prepare.

### SUMMARY

The present disclosure provides a pharmaceutical composition. The pharmaceutical composition comprises an active ingredient and a pharmaceutically acceptable auxiliary material;

the active ingredient comprises cabotegravir or a pharmaceutically acceptable salt thereof, and the auxiliary material comprises a surfactant and a suspending agent.

According to an embodiment of the present disclosure, the pharmaceutical composition further comprises a lyophilization excipient.

According to an embodiment of the present disclosure, the suspending agent is a cellulose suspending agent, for example, selected from at least one of carboxymethylcellulose and a sodium salt thereof, hydroxypropylcellulose and a sodium salt thereof, hydroxypropyl methylcellulose and a sodium salt thereof, methylcellulose and a sodium salt thereof, hydroxyethylcellulose and a sodium salt thereof, sodium hyaluronate, and polyvinylpyrrolidone, for example, selected from at least one of methylcellulose, sodium carboxymethylcellulose, and hydroxypropylcellulose.

According to an embodiment of the present disclosure, the surfactant is selected from at least one of polysorbate or a derivative thereof, and polyethylene glycol stearate or a derivative thereof; for example, the polyethylene glycol stearate or the derivative thereof is selected from polyethylene glycol 15-hydroxystearate, and the polysorbate or the derivative thereof is selected from polysorbate-80. Preferably, the surfactant is selected from at least one of polyethylene glycol 15-hydroxystearate and polysorbate-80.

According to an embodiment of the present disclosure, the lyophilization excipient is selected from at least one of mannitol, trehalose, glucose, and the like.

According to an embodiment of the present disclosure, the auxiliary material may further comprise an isoosmotic adjusting agent. For example, the isoosmotic adjusting agent may be selected from one or more of sodium chloride, mannitol, sorbitol, glucose, sucrose, fructose, and lactose. In one embodiment, the isoosmotic adjusting agent may be sodium chloride and mannitol. In another embodiment, the isoosmotic adjusting agent may be sodium chloride. In yet another embodiment, the isoosmotic adjusting agent may be mannitol.

According to an embodiment of the present disclosure, the surfactant and cabotegravir or the pharmaceutically acceptable salt thereof are in a weight ratio selected from 1:(0.01-100), such as 1:(1-100), preferably 1:(10-70), and more preferably 1:(50-70). As an example, the surfactant and cabotegravir or the pharmaceutically acceptable salt thereof are in a weight ratio of 1:6.67, 1:10, 1:20, 1:40, 1:42, 1:60, or 1:66.67.

According to an embodiment of the present disclosure, the suspending agent and cabotegravir or the pharmaceutically acceptable salt thereof are in a weight ratio selected from 1:(0.01-100), such as 1:(1-100), preferably 1:(2.5-50), and more preferably 1:(25-30). As an example, the suspending agent and cabotegravir or the pharmaceutically acceptable salt thereof are in a weight ratio of 1:2, 1:2.86, 1:2.5, 1:10, 1:25, or 1:26.

According to an embodiment of the present disclosure, the lyophilization excipient and cabotegravir or the pharmaceutically acceptable salt thereof are in a weight ratio selected from 1:(0.01-100), such as 1:(0.1-50), preferably 1:(0.1-20). As an example, the lyophilization excipient and cabotegravir or the pharmaceutically acceptable salt thereof are in a weight ratio of 1:0.5, 1:1, 1:5, or 1:5.9.

In one embodiment, the pharmaceutical composition comprises 0.01-800 mg, such as 10-500 mg, preferably 100-400 mg, and more preferably 200-400 mg of cabotegravir or a pharmaceutically acceptable salt thereof; as an example, the pharmaceutical composition comprises 40 mg. 100 mg, 200 mg. 300 mg. 400 mg. 423.7 mg, or 450 mg of cabotegravir or a pharmaceutically acceptable salt thereof.

In one embodiment, the active ingredient is cabotegravir or a sodium salt thereof.

According to one embodiment of the present disclosure, the pharmaceutical composition is a powder, preferably an injectable powder; as an example, the pharmaceutical composition is an injectable lyophilized powder.

According to an exemplary embodiment of the present disclosure, the pharmaceutical composition comprises cabotegravir, polysorbate-80, sodium carboxymethylcellulose, and mannitol in a weight ratio of (5-70):1:(1-10):(5-30), such as (6-68):1:(1.5-5):(10-25), and illustratively 10:1:3.5:20, 6.67:1:2.67:20, or 66.67:1:3.5:20.

According to an exemplary embodiment of the present disclosure, the pharmaceutical composition comprises cabotegravir or a sodium salt thereof, polyethylene glycol 15-hydroxystearate, sodium carboxymethylcellulose, and mannitol in a weight ratio of (5-70):1:(1-10):(5-30), such as (6-68): 1:(1.5-5):(7-15), and illustratively 40:1:1.6:8.

According to an embodiment of the present disclosure, the pharmaceutical composition may further comprise a second therapeutic agent selected from an HIV inhibitor; for example, the HIV inhibitor may be selected from one or more of a nucleoside reverse transcriptase inhibitor, a non-nucleoside reverse transcriptase inhibitor, a protease inhibitor, and an integrase inhibitor.

The present disclosure further provides a pharmaceutical formulation comprising the pharmaceutical composition.

In some embodiments, the pharmaceutical formulation comprises the pharmaceutical composition and a dispersing solvent. In some embodiments, the pharmaceutical formulation is prepared by suspending the pharmaceutical composition in the dispersing solvent, preferably by suspending an injectable lyophilized powder in the dispersing solvent.

According to an embodiment of the present disclosure, the dispersing solvent is water.

According to an embodiment of the present disclosure, cabotegravir or the pharmaceutically acceptable salt thereof has a concentration selected from 0.01-800 mg/mL, such as 10-400 mg/mL, preferably 20-400 mg/mL, and more preferably 150-200 mg/mL.

According to an embodiment of the present disclosure, in the pharmaceutical formulation, particles of the pharmaceutical composition in the dispersing solvent have particle size distributions as follows: D10 is in the range of about 0.5 μm to about 10 μm, such as 1, 2, 3, 4, 5, 6, 7, 8, 9, or 10 μm; D50 is in the range of about 2 μm to about 25 μm, such as 2, 3, 4, 5, 6, 7, 8, 9, 10, 11, 12, 13, 14, 15, 16, 17, 18, 19, 20, 21, 22, 23, 24, or 25 μm; a particle size D90 is in the range of about 5 μm to about 50 μm, such as 6, 7, 8, 9, 10, 11, 12, 13, 14, 15, 16, 17, 18, 19, 20, 25, 30, 35, 40, or 45 μm.

In some embodiments, the D50 has a change range of less than about 2 μm, such as a change range of 0.1, 0.3, 0.5, 0.7, 1.0, 1.2, 1.5, or 1.8 μm, under a storage condition of room temperature or high temperature (50-70° C., such as 60)° ° C.; the D90 has a change range of less than about 5 μm, such as a change range of 0.5, 1.0, 1.5, 2.0, 2.5, 3.0, 3.5, 4.0, or 4.5 μm. In some embodiments, all of the D10, D50 and D90 have a change range of less than 1 μm, such as independently, a change range of 0.1, 0.2, 0.3, 0.4, 0.5, 0.6, 0.7, 0.8, or 0.9 μm, under an accelerated experiment condition of room temperature or high temperature (50-70° C., such as 60° C.).

Further, the storage time may be 1 month, 2 months, 3 months or more.

In one embodiment, the D50 of the particles has a change range of less than about 2 μm, such as a change range of 0.1, 0.3, 0.5, 0.7, 1.0, 1.2, 1.5, or 1.8 μm during storage at room temperature for 2 months; the D90 has a change range of less than about 5 μm, such as a change range of 0.5, 1.0, 1.5, 2.0, 2.5, 3.0, 3.5, 4.0, or 4.5 μm.

According to an embodiment of the present disclosure, the particle size D50 of the particles is less than 30 μm, preferably less than 20 μm, and more preferably less than 5 μm. For example, the particle size D50 is 1, 2, 3, 4, 5, 6, 7, 8, 9, 10, 11, 12, 13, 14, 15, 16, 17, 18, 19, 20, 21, 22, 23, 24, or 25 μm.

According to an embodiment of the present disclosure, the pharmaceutical formulation is an injection, such as a suspension injection.

According to an embodiment of the present disclosure, the unit dosage form of the pharmaceutical formulation comprises 20-60 mg, such as 30 mg. 40 mg, or 50 mg of cabotegravir, and the content calculated by cabotegravir is a therapeutically effective amount.

According to an embodiment of the present disclosure, the pharmaceutical formulation may be administered by subcutaneous injection or intramuscular injection.

According to an embodiment of the present disclosure, the pharmaceutical formulation is used for preventing and/or treating a disease caused by human immunodeficiency virus (HIV) infection.

The present disclosure further provides a preparation method for the pharmaceutical composition, which comprises the following steps: mixing an organic solution containing cabotegravir or the pharmaceutically acceptable salt thereof with water, filtering and washing to obtain a precipitate, mixing the obtained precipitate with an auxiliary material, and performing lyophilization.

According to an embodiment of the present disclosure, the organic solvent in the organic solution containing cabotegravir or the pharmaceutically acceptable salt thereof is selected from at least one of DMF, DMA, DMSO, and acetic acid.

According to an embodiment of the present disclosure, the organic solution containing cabotegravir or the pharmaceutically acceptable salt thereof is mixed with water by shearing or stirring at a rotation speed of 100-10000 rpm (e.g., 600-8000 rpm, such as 3500-5000 rpm).

According to a preferred embodiment of the present disclosure, the preparation method comprises the following steps:
(1) dissolving cabotegravir or the pharmaceutically acceptable salt thereof in an organic solvent to obtain a first mixture;
(2) pouring the first mixture into water under dispersive conditions to obtain a second mixture;
(3) filtering and washing the second mixture to obtain cabotegravir;
(4) homogeneously mixing cabotegravir obtained in step (3) with the pharmaceutically acceptable auxiliary material described above; and
(5) after the completion of step (4), performing lyophilization.

According to an embodiment of the present disclosure, in step (2), the water temperature is controlled at 0-100° C. preferably 0-60° C., such as 15° C., 25° C., or 50° C.

Optionally, the preparation method further comprises further adjusting the particle sizes before the lyophilization by homogenization, microfluidization and the like according to actual situations.

The present disclosure further provides a preparation method for the pharmaceutical formulation described above, which comprises suspending the pharmaceutical composition described above in a dispersing solvent, preferably suspending the lyophilized pharmaceutical composition prepared by the above method in a dispersing solvent.

### Definitions and Explanations of Terms

The term “pharmaceutically acceptable salt” refers to a salt that retains the desired biological activity of the compound of the present subject matter and exhibits minimal undesired toxicological effects. These pharmaceutically acceptable salts may be prepared in situ during the final separation and purification of the compounds, or prepared by subjecting the purified compounds in their free acid or free base forms to separate reactions with suitable bases or acids, respectively.

The suitable pharmaceutically acceptable salts may include acid or base addition salts. The representative pharmaceutically acceptable base addition salts include, but are not limited to, aluminum, 2-amino-2-(hydroxymethyl)-1,3-propanediol (TRIS, tromethamine), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p-chlorobenzyl-2-pyrrolidin-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, tert-butylamine, and zinc.

Unless otherwise stated, all numbers expressing quantities, concentrations, ratios, weights, volumes, rotation speeds, pressures, particle sizes, percentages, technical effects, and the like used in the specification and claims are to be understood as being modified in any instances by the term “about” or “approximately”. “About” represents a range of +10% of the numerical value modified thereby.

Accordingly, unless indicated to the contrary, the numerical parameters set forth in the following specification and appended claims are approximations. Unless otherwise stated, the terms used herein have the meanings that are commonly understood by those skilled in the art. It should be understood by those skilled in the art that the numerical parameters can vary depending on the desired properties and effects sought and obtained by the present disclosure, and each numerical parameter should be interpreted according to the number of significant digits and conventional rounding rules or as understood by those skilled in the art.

Although the numerical ranges and parameters setting forth the broad scope of the present disclosure are approximations, the numerical values set forth in the specific examples are provided as precisely as possible. However, any numerical value inherently contains certain errors necessarily resulting from the standard deviation found in their respective tests or measurements. Each numerical range given in this specification will include each narrower numerical range that falls within this broader numerical range, as if such narrower numerical ranges are all explicitly written herein.

The term “stable” related to the particle size means that D10 is in the range of about 0.5 μm to about 10 μm, D50 is in the range of about 2 μm to about 25 μm, and the particle size D90 is in the range of about 5 μm to about 50 μm; meanwhile, the D50 has a change range of less than about 2 μm, and the D90 has a change range of less than about 5 μm. When the cabotegravir suspension injection has a stable particle size, it can not only achieve the effect of long-term sustained release, but also make the plasma concentration stable, and facilitate clinical administration.

The term “particularly stable” related to the particle size means that D10 is in the range of about 0.5 μm to about 10 μm, D50 is in the range of about 2 μm to about 25 μm, and D90 is in the range of about 5 μm to about 50 μm; meanwhile, all of the D10, D50 and D90 have a change range of less than 1 μm.

When the suspension injection obtained by reconstituting the cabotegravir powder formulation has a particularly stable particle size, it can further achieve the long-term sustained release, make the plasma concentration stable, and facilitate clinical administration.

The term “median particle size D50” or “D50” refers to a particle size at 50% of a cumulative particle size distribution. That is, particles having a smaller size than this particle size account for 50% of all particles by volume or content.

The term “D10” refers to a particle size at 10% of a cumulative particle size distribution. That is, particles having a smaller size than this particle size account for 10% of all particles by volume or content.

The term “D90” refers to a particle size at 90% of a cumulative particle size distribution. That is, particles having a smaller size than this particle size account for 90% of all particles by volume or content.

The term “at least one” refers to two, three or more.

The term “more” in “one or more” refers to two, three or more.

### Beneficial Effects

The pharmaceutical composition provided in the present disclosure relates to use of the pharmaceutical composition for the treatment and prevention of HIV infection, has good stability, and is suitable for industrial production.

The present disclosure provides an injectable pharmaceutical composition/lyophilized formulation containing cabotegravir, which can be stored at room temperature and has long storage period. After reconstituting the lyophilized powder formulation, the particle size of the obtained cabotegravir suspension injection always remains stable, even to a particularly stable degree.

The present disclosure further provides a preparation method for the cabotegravir injection, which has a relatively simple production process and relatively low prices of starting materials and production equipment, and is easy to achieve the expansion of the yield, compared with the original preparation method of dispersion and milling.

The present disclosure further provides a cabotegravir suspension injection, which can maintain the steady-state concentration for a longer time compared with the original nanosuspension injection, so that the administration frequency can be reduced.

### BRIEF DESCRIPTION OF THE DRAWINGS

FIG. 1 shows particle size distributions of cabotegravir suspension injections of different formulas (i.e., after reconstitution of lyophilized powders);

FIG. 2 shows a plasma concentration-time curve for the administration by intramuscular injection in Example 10.

### DETAILED DESCRIPTION

The technical scheme of the present disclosure will be further illustrated in detail with reference to the following specific examples. It should be understood that the following embodiments are merely exemplary illustration and explanation of the present disclosure, and should not be construed as limiting the protection scope of the present disclosure. All techniques implemented based on the content of the present disclosure described above are encompassed within the protection scope of the present disclosure.

Unless otherwise stated, the starting materials and reagents used in the following examples are all commercially available products or can be prepared using known methods.

In terms of the starting materials and reagents used in the examples, cabotegravir was purchased from Shanghai Desano Pharmaceuticals Co., Ltd., sodium carboxymethylcellulose was purchased from Ashland, polysorbate-80 was purchased from Nanjing Well Pharmaceutical Co., Ltd., mannitol was purchased from Roquette (France), NMP was purchased from RHAWN, DMA (N,N-dimethylacetamide) was purchased from Finar Limited Company, DMSO was purchased from Emanuel Merck, Darmstadt, and acetic acid was purchased from Sinopharm Chemical Reagent Co., Ltd. Unless otherwise stated, the reaction temperature in each step was room temperature and the pressure was normal pressure. The particle sizes of the suspensions in the examples were detected using a laser particle analyzer (model: HELOs) manufactured by SYMPATEC GmbH.

In the following examples, the crystallization temperature of the cabotegravir was room temperature unless otherwise stated.

### Example 1: Cabotegravir Suspension Injection (Lyophilized Powder) I and Preparation Thereof

TABLE 1
Cabotegravir suspension injection (lyophilized powder) I

| | | Formula amount in | |
|---|---|---|---|
| Component | Function | a single dose | |
| Cabotegravir | Active ingredient | 40 | mg |
| Polysorbate-80 | Surfactant | 4 | mg |
| Sodium | Suspending agent | 14 | mg |
| carboxymethylcellulose | | | |
| Mannitol | Lyophilization excipient | 80 | mg |

Preparation method: cabotegravir was dissolved with NMP of 15 times the weight of cabotegravir at 60-70° C. The agent liquid was added to water of 10 times the volume of NMP at a uniform speed within 10 min under the stirring condition of 600 rpm to obtain cabotegravir by precipitation. The mixture solution was filtered. The precipitate was washed with water, and then homogeneously mixed with auxiliary materials in Table 1 and ultrapure water. The mixture was lyophilized to obtain the cabotegravir suspension injection (lyophilized powder) I. 2 mL of water for injection was added for reconstitution, then the particle sizes were detected to be D10 of 2.00 μm, D50 of 8.25 μm, and D90 of 18.99 μm.

### Example 2: Cabotegravir Suspension Injection (Lyophilized Powder) II and Preparation Thereof

TABLE 2
Cabotegravir suspension injection (lyophilized powder) II

| | | Formula amount in | |
|---|---|---|---|
| Component | Function | a single dose | |
| Cabotegravir | Active ingredient | 400 | mg |
| Polysorbate-80 | Surfactant | 6 | mg |
| Sodium | Suspending agent | 16 | mg |
| carboxymethylcellulose | | | |
| Mannitol | Lyophilization excipient | 80 | mg |

Preparation method: cabotegravir was dissolved with DMSO of 15 times the weight of cabotegravir at 60-70° ° C. The agent liquid was added to water of 10 times the volume of DMSO (ice water bath, controlling the crystallization temperature below 15° C.) within 10 min under the shearing condition of 5000 rpm to obtain cabotegravir by precipitation. The mixture solution was filtered. The precipitate was washed with water, and then homogeneously mixed with auxiliary materials in Table 2 and ultrapure water. The mixture was lyophilized to obtain the cabotegravir suspension injection (lyophilized powder) II. 2 mL of water for injection was added for reconstitution, then the particle sizes were detected to be D10 of 1.43 μm, D50 of 5.33 μm, and D90 of 12.89 μm.

### Example 3: Cabotegravir Suspension Injection (Lyophilized Powder) III and Preparation Thereof

TABLE 3
Cabotegravir suspension injection (lyophilized powder) III

| | | Formula amount in | |
|---|---|---|---|
| Component | Function | a single dose | |
| Cabotegravir | Active ingredient | 400 | mg |
| Polyethylene glycol 15- | Surfactant | 10 | mg |
| hydroxystearate | | | |
| Sodium | Suspending agent | 16 | mg |
| carboxymethylcellulose | | | |
| Mannitol | Lyophilization excipient | 80 | mg |

The specific operation steps were the same as those in Example 2. The mixture was lyophilized to obtain the cabotegravir suspension injection (lyophilized powder) III. 2 mL of water for injection was added for reconstitution, then the particle sizes were detected to be D10 of 1.23 μm, D50 of 4.13 μm, and D90 of 9.94 μm.

### Example 4: Cabotegravir Suspension Injection (Lyophilized Powder) IV and Preparation Thereof

TABLE 4
Cabotegravir suspension injection (lyophilized powder) IV

| | | Formula amount in | |
|---|---|---|---|
| Component | Function | a single dose | |
| Cabotegravir sodium | Active ingredient | 423.7 | mg |
| Polyethylene glycol 15- | Surfactant | 10 | mg |
| hydroxystearate | | | |
| Sodium | Suspending agent | 16 | mg |
| carboxymethylcellulose | | | |
| Mannitol | Lyophilization excipient | 72 | mg |

Note:
the single dose of cabotegravir sodium converted into cabotegravir was 400 mg.

Preparation method: cabotegravir sodium was dissolved with acetic acid of 10 times the weight of cabotegravir sodium at room temperature. The agent liquid was added to water of 4 times the volume of acetic acid within 10 min under the shearing condition of 5000 rpm to obtain cabotegravir by precipitation. The mixture solution was filtered. The precipitate was washed with water, and then homogeneously mixed with auxiliary materials in Table 4 and ultrapure water. The mixture was lyophilized to obtain the cabotegravir suspension injection (lyophilized powder) IV. 2 mL of water for injection was added for reconstitution, then the particle sizes were detected to be D10 of 7.97 μm, D50 of 22.32 μm, and D90 of 45.18 μm.

### Example 5: Cabotegravir Suspension Injection (Lyophilized Powder) V and Preparation Thereof

TABLE 5
Cabotegravir suspension injection (lyophilized powder) V

| | | Formula amount in | |
|---|---|---|---|
| Component | Function | a single dose | |
| Cabotegravir sodium | Active ingredient | 423.7 | mg |
| Polyethylene glycol 15- | Surfactant | 10 | mg |
| hydroxystearate | | | |
| Sodium | Suspending agent | 16 | mg |
| carboxymethylcellulose | | | |
| Mannitol | Lyophilization excipient | 72 | mg |

Note:
the single dose of cabotegravir sodium converted into cabotegravir was 400 mg.

Preparation method: cabotegravir was dissolved with acetic acid of 10 times the weight of cabotegravir at room temperature. The agent liquid was added to water of 4 times the volume of acetic acid within 10 min under the shearing condition of 5000 rpm to obtain cabotegravir by precipitation. The mixture solution was filtered. The precipitate was washed with water, and then homogeneously mixed with auxiliary materials in Table 5 and ultrapure water. The agent liquid was homogenized using a homogenizer 3 times at a pressure of 1900 bar, and then lyophilized to obtain the cabotegravir suspension injection (lyophilized powder) V. 2 mL of water for injection was added for reconstitution, then the particle size was detected to be D10 of 1.20 μm, D50 of 3.54 μm, and D90 of 9.01 μm.

Cabotegravir sodium was dissolved with acetic acid to produce cabotegravir, which was prepared into the cabotegravir suspension injection. Cabotegravir could also be dissolved with acetic acid, and prepared into the cabotegravir suspension injection by the same process.

In addition, the particle sizes of the present product could be further adjusted before lyophilization by homogenization, microfluidization and the like.

### Example 6: Cabotegravir Suspension Injection (Lyophilized Powder) VI and Preparation Thereof

TABLE 6
Cabotegravir suspension injection (lyophilized powder) VI

| | | Formula amount in | |
|---|---|---|---|
| Component | Function | a single dose | |
| Cabotegravir | Active ingredient | 400 | mg |
| Poloxamer 188 | Surfactant | 10 | mg |
| Sodium | Suspending agent | 16 | mg |
| carboxymethylcellulose | | | |
| Mannitol | Lyophilization excipient | 80 | mg |

The specific operation steps were the same as those in Example 2. The mixture was lyophilized to obtain the cabotegravir suspension injection (lyophilized powder) VI. 2 mL of water for injection was added for reconstitution, then the particle size was detected to be D10 of 2.00 μm, D50 of 8.47 μm, and D90 of 35.18 μm.

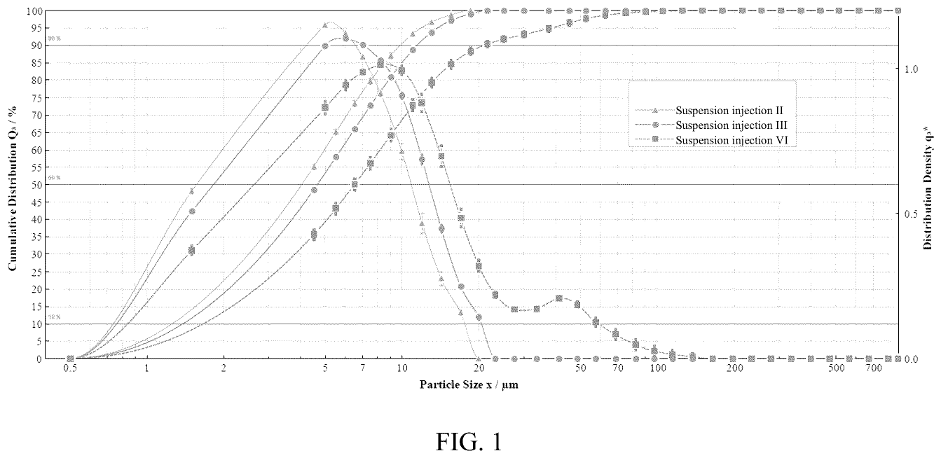

The particle size distribution results in FIG. 1 show that the cabotegravir suspension injection (lyophilized powder) VI containing poloxamer 188 as the surfactant has distinctly bimodal particle size distribution after reconstitution, while the other formulas have unimodal particle size distributions. Therefore, HS15 (polyethylene glycol 15-hydroxystearate) and tween 80 are preferably selected as the surfactant of the present product.

### Example 7: Formula Stability Investigation

The lyophilized powders of suspension injections of all formulas were stored at room temperature or high temperature for a period of time and then reconstituted. The particle size was detected. The results are shown in Table 7. The particle size results of the non-lyophilized cabotegravir suspension after being stored at room temperature for 7 days are shown in Table 7.

TABLE 7
Particle size stability data of all formulas

| Name of formula | Storage time | D(10)/μm | D(50)/μm | D(90)/μm |
|---|---|---|---|---|
| Cabotegravir suspension | Reconstitution on day 0 | 2.00 | 8.25 | 18.99 |
| injection (lyophilized | Non-lyophilized suspension | 7.53 | 18.22 | 48.87 |
| powder) I | was stored at room | | | |
| | temperature for 7 days | | | |
| | Lyophilized powder was | 1.83 | 7.56 | 18.34 |
| | reconstituted after being | | | |
| | stored at room temperature | | | |
| | for 3 months | | | |
| Cabotegravir suspension | Lyophilized powder was | 1.43 | 5.33 | 12.89 |
| injection (lyophilized | reconstituted after being | | | |
| powder) II | stored at room temperature | | | |
| | for 0 day | | | |
| | Lyophilized powder was | 1.45 | 5.39 | 12.90 |
| | reconstituted after being | | | |
| | stored at room temperature | | | |
| | for 1 month | | | |
| Cabotegravir suspension | Reconstitution on day 0 | 1.23 | 4.13 | 9.94 |
| injection (lyophilized | Lyophilized powder was | 1.82 | 4.78 | 12.24 |
| powder) III | reconstituted after being | | | |
| | stored at room temperature | | | |
| | for 2 months | | | |
| | Lyophilized powder was | 1.73 | 4.77 | 10.24 |
| | reconstituted after being | | | |
| | stored at room temperature | | | |
| | for 3 months | | | |
| | Lyophilized powder was | 1.66 | 4.11 | 10.66 |
| | reconstituted after being | | | |
| | stored at 60° C. for 4 days | | | |
| | Lyophilized powder was | 1.60 | 5.62 | 15.92 |
| | reconstituted after being | | | |
| | stored at 60° C. for 10 days | | | |
| Cabotegravir suspension | Reconstitution on day 0 | 7.97 | 22.32 | 45.18 |
| injection (lyophilized | Lyophilized powder was | 8.01 | 22.65 | 45.53 |
| powder) IV | reconstituted after being | | | |
| | stored at room temperature | | | |
| | for 1 day | | | |
| | Lyophilized powder was | 7.79 | 22.29 | 45.00 |
| | reconstituted after being | | | |
| | stored at room temperature | | | |
| | for 2 months | | | |
| Cabotegravir suspension | Reconstitution on day 0 | 1.20 | 3.54 | 9.01 |
| injection (lyophilized | Lyophilized powder was | 1.23 | 3.86 | 11.89 |
| powder) V | reconstituted after being | | | |
| | stored at room temperature | | | |
| | for 2 months | | | |

As can be seen from the data in Table 7, all of the cabotegravir suspension injections (lyophilized powder) I-V were relatively stable under the above conditions, and the particle size distributions had no significant change compared with the initial one. In Example 1, after the non-lyophilized suspension was stored at room temperature for 7 days, the particle sizes of D10, D50 and D90 were significantly increased compared with those of the lyophilized powders after being reconstituted. This suggests that the particle size stability of the reconstituted suspension injection after lyophilization is significantly superior to that of the non-lyophilized suspension.

### Example 8: Cabotegravir Suspension Injection (Lyophilized Powder) VII and Preparation Thereof

The formula was the same as that in Example 2, the temperature of the agent liquid was above 50° C. when the crystallization was performed in the process, and the other steps were the same as those in Example 2. Finally, the sample with the particle sizes of D10 of 9.01 μm, D50 of 19.92 μm, and D90 of 43.16 μm after reconstitution was obtained, and used for animal experiments.

### Example 9: Cabotegravir Nanosuspension Injection VIII and Preparation Thereof

TABLE 8
Cabotegravir nanosuspension injection VIII

| | | Formula amount in | |
|---|---|---|---|
| Component | Function | a single dose | |
| Cabotegravir | Active ingredient | 400 | mg |
| Polysorbate-20 | Surfactant | 40 | mg |
| Polyethylene glycol | Stabilizer | 40 | mg |
| (PEG) 3350 | | | |
| Mannitol | isoosmotic adjusting agent | 70 | mg |

According to the formula in the FDA review document for the original formulation Cabenuva published by ViiV Healthcare, and the preferred particle size of less than or equal to 200 nm in the claims of the original patent WO2012/037320, the cabotegravir nanosuspension injection VIII was prepared with the formula amount of the starting material and auxiliary materials by milling process; the milling equipment was DYNO®-MILL RESEARCHLAB (ceramic accelerator, Ø 0.2 mm (TOSOH) ZrO₂ microbeads). The sample with the particle size distribution of D10 of 60.6 nm, D50 of 139 nm, and D90 of 489 nm was obtained after milling, and used as the control formulation in the animal experiments.

### Example 10: Pharmacokinetic Study in Rats

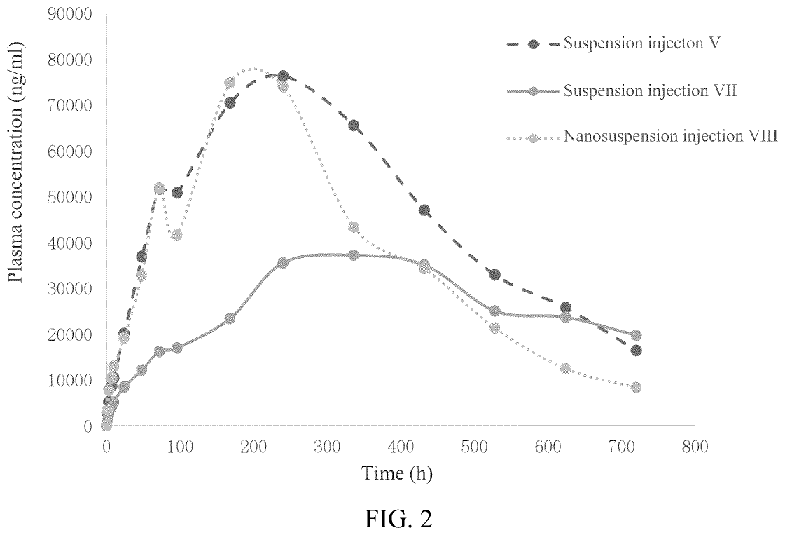

SD rats (4 rats/group, 2 male and 2 female) were administered the following suspension injections by intramuscular (IM) injection in a single dose: cabotegravir suspension injection V, suspension injection VII nanosuspension injection VIII, all at a dose of 20 mg/kg. After administration, the rats were maintained for 720 h without treatment. FIG. 2 shows the mean concentration-time curve of cabotegravir in plasma after the intramuscular injection of different formulations to the rats.

After the intramuscular injection of the cabotegravir suspension injection VII to the rats, the drug concentration-time curve of the cabotegravir in plasma was relatively flat, and the peak concentration was lower than that of the control formulation (i.e., the nanosuspension injection VIII). The plasma concentration maintained was higher than that of the control formulation from 528 h after administration. The concentration was still at a relatively higher level by the last time point (720 h) due to the limitation of the sampling time point.

After the intramuscular injection of the cabotegravir suspension injection V to the rats, the concentration of cabotegravir in plasma increased and reached a level equivalent to that of the control formulation at about 250 h. Then, the concentration of cabotegravir in plasma decreased slowly, maintaining a plasma concentration higher than that of the control formulation. The concentration was still at a relatively higher level by the last time point (720 h).

According to the PK experiment results shown as in table 9, the suspension injection VII (D50 of about 20 μm) has relatively lower Cₘₐₓ but can maintain a steady-state concentration for a longer period of time compared with the control formulation (D50 of about 150 nm), so the suspension injection VII has a better sustained-release effect. The suspension injection V (D50 of about 3.5 μm) can be released rapidly to reach the Cₘₐₓ equivalent to that of the control formulation, and the plasma concentration is slowly decreased after reaching the peak value, so the suspension injection V has a stronger sustained-release effect. The original formulation was required to be injected once a month to maintain the steady-state concentration, while the present product could reduce the administration frequency and could maintain the steady state even if administered at longer intervals.

TABLE 9
Pharmacokinetic parameters of cabotegravir in plasma after
intramuscular injection of suspension injection V, suspension
injection VII and nanosuspension injection VIII to rats

| | | Suspension | Nanosuspension | Suspension |
|---|---|---|---|---|
| i.m. | | injection VII | injection VIII | injection V |
| Administration dose | | 20 mg/kg | | |
| t_{1/2} | h | 654.6 | 148.7 | 225.4 |
| Tₘₐₓ | h | 336 | 204 | 246 |
| Cₘₐₓ | ng/mL | 34770 | 81170 | 81023 |
| AUCₗₐₛₜ | h*ng/mL | 14480000 | 27260000 | 33772474 |
| AUC_{inf} | h*ng/mL | 35160000 | 29040000 | 39225736 |
| Vz_F_obs | mL/kg | 346.6 | 150.2 | 170 |
| Cl_F_obs | mL/h/kg | 0.4126 | 0.6962 | 0.519 |
| MRTₗₐₛₜ | h | 297.9 | 280.5 | 315.8 |

The embodiments of the present disclosure have been described above. However, the present disclosure is not limited to the embodiments described above. Any modification, equivalent, improvement and the like made without departing from the spirit and principle of the present disclosure shall fall within the protection scope of the present disclosure.

1. A pharmaceutical composition, wherein the pharmaceutical composition comprises an active ingredient and a pharmaceutically acceptable auxiliary material;
the active ingredient comprises cabotegravir or a pharmaceutically acceptable salt thereof, and the auxiliary material comprises a surfactant and a suspending agent.

2. The pharmaceutical composition according to claim 1, wherein the pharmaceutical composition further comprises a lyophilization excipient.

3. The pharmaceutical composition according to claim 2, wherein the suspending agent is a cellulose suspending agent, for example, selected from at least one of carboxymethylcellulose and a sodium salt thereof, hydroxypropylcellulose and a sodium salt thereof, hydroxypropyl methylcellulose and a sodium salt thereof, methylcellulose and a sodium salt thereof, hydroxyethylcellulose and a sodium salt thereof, sodium hyaluronate, and polyvinylpyrrolidone; and/or
the surfactant is selected from at least one of polysorbate or a derivative thereof, and polyethylene glycol stearate or a derivative thereof; for example, the polyethylene glycol stearate or the derivative thereof is selected from polyethylene glycol 15-hydroxystearate, and the polysorbate or the derivative thereof is selected from polysorbate-80; and/or
the lyophilization excipient is selected from at least one of mannitol, trehalose, and glucose;
preferably, the auxiliary material further comprises an isoosmotic adjusting agent.

4. The pharmaceutical composition according to claim 2, wherein the surfactant and cabotegravir or the pharmaceutically acceptable salt thereof are in a weight ratio selected from 1:(0.01-100); and/or
the suspending agent and cabotegravir or the pharmaceutically acceptable salt thereof are in a weight ratio selected from 1:(0.01-100); and/or
the lyophilization excipient and cabotegravir or the pharmaceutically acceptable salt thereof are in a weight ratio selected from 1:(0.01-100).

5. The pharmaceutical composition according to claim 1, wherein the pharmaceutical composition is a powder, preferably an injectable powder, and more preferably an injectable lyophilized powder;
preferably, the pharmaceutical composition further comprises a second therapeutic agent selected from an HIV inhibitor.

6. A preparation method for the pharmaceutical composition according to claim 1, wherein the preparation method comprises the following steps: mixing an organic solution containing cabotegravir or the pharmaceutically acceptable salt thereof with water, filtering and washing to obtain a precipitate, mixing the obtained precipitate with an auxiliary material, and performing lyophilization.

7. A pharmaceutical formulation, wherein the pharmaceutical formulation comprises the pharmaceutical composition according to claim 1;
preferably, the pharmaceutical formulation comprises the pharmaceutical composition and a dispersing solvent;
preferably, the pharmaceutical formulation is prepared by suspending the pharmaceutical composition in the dispersing solvent, preferably by suspending an injectable lyophilized powder in the dispersing solvent;
preferably, the dispersing solvent is water; and
preferably, cabotegravir or the pharmaceutically acceptable salt thereof has a concentration selected from 0.01-800 mg/mL.

8. The pharmaceutical formulation according to claim 7, wherein in the pharmaceutical formulation, particles of the pharmaceutical composition in the dispersing solvent have particle size distributions as follows: D10 is in the range of about 0.5 μm to about 10 μm, D50 is in the range of about 2 μm to about 25 μm, and a particle size D90 is in the range of about 5 μm to about 50 μm.

9. The pharmaceutical formulation according to claim 8, wherein the pharmaceutical formulation is an injection, such as a suspension injection;
preferably, the pharmaceutical formulation is used for preventing and/or treating a disease caused by human immunodeficiency virus infection.

10. A preparation method for the pharmaceutical formulation according to claim 7, comprising suspending a pharmaceutical composition in a dispersing solvent, wherein the pharmaceutical composition comprises an active ingredient and a pharmaceutically acceptable auxiliary material;
the active ingredient comprises cabotegravir or a pharmaceutically acceptable salt thereof, and the auxiliary material comprises a surfactant and a suspending agent;
preferably, the dispersing solvent is water; and
preferably, cabotegravir or the pharmaceutically acceptable salt thereof has a concentration selected from 0.01-800 mg/mL.

11. The preparation method according to claim 10, wherein the pharmaceutical composition further comprises a lyophilization excipient.

12. The pharmaceutical method according to claim 11, wherein the suspending agent is a cellulose suspending agent, for example, selected from at least one of carboxymethylcellulose and a sodium salt thereof, hydroxypropylcellulose and a sodium salt thereof, hydroxypropyl methylcellulose and a sodium salt thereof, methylcellulose and a sodium salt thereof, hydroxyethylcellulose and a sodium salt thereof, sodium hyaluronate, and polyvinylpyrrolidone; and/or
the surfactant is selected from at least one of polysorbate or a derivative thereof, and polyethylene glycol stearate or a derivative thereof; for example, the polyethylene glycol stearate or the derivative thereof is selected from polyethylene glycol 15-hydroxystearate, and the polysorbate or the derivative thereof is selected from polysorbate-80; and/or
the lyophilization excipient is selected from at least one of mannitol, trehalose, and glucose;
preferably, the auxiliary material further comprises an isoosmotic adjusting agent.

13. The pharmaceutical method according to claim 11, wherein the preparation method comprises the following steps:
(1) dissolving cabotegravir or the pharmaceutically acceptable salt thereof in an organic solvent to obtain a first mixture;
(2) pouring the first mixture into water under dispersive conditions to obtain a second mixture;
(3) filtering and washing the second mixture to obtain cabotegravir;
(4) homogeneously mixing cabotegravir obtained in step (3) with the pharmaceutically acceptable auxiliary material described above; and
(5) after the completion of step (4), performing lyophilization.
preferably, the water temperature is controlled at 0-100° C., preferably 0-60° C.;
preferably, the preparation method further comprises adjusting the particle sizes before the lyophilization by homogenization, microfluidization and the like.
